SEQUENCE LISTING (1 sequence)

SEQ ID NO: 1 - protein, 572 aa, Babesia bovis
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVEEEETDEYPIEVEEPYDELLEEANVQVHGAPNAMSLNLLMPVNTHRILKTSEFDSAHTPVHYKPLFPFKLVSVSWSESPIFEIPVPEEEQGKEEKTEETETKSEEDGEEAEGADEAAPAILHADLQNKFIDEVVVFRNCFDTAVSVNVDGKQIYFTATGNEAEDFEEVEADEYEKFMNKIAKTFTIDISETDLKSECLVAEDDKVYDLSWYSIHAAPCYLADKVLFKGNPLWEATDKKEHFAGCSVINDGDDRLIALSIRNGPENRQIYFYGVDGALREIDNDEMVTHYEAFKEKEQAERERYEAEEKARLEAEKDAEKEEEENKEGAEEDEEEEEEVAEEEEITADIEE

(19) United States
(12) Patent Application Publication
(10) Pub. No.: US 2019/0271697 A1
(43) Pub. Date: Sep. 5, 2019

(54) Modified Indirect Enzyme Linked Immunosorbent Assay Optimal for Monitoring Acute and Long Term Carrier Infections of Diverse Babesia bovis Strains

(71) Applicants: The United States of America, as Represented by the Secretary of Agriculture, Washington, DC (US); VMRD INC and Washington State University, Pullman, WA (US); VMRD Inc., Pullman, WA (US)

(72) Inventors: Carlos E. Suarez, Pullman, WA (US); Chungwon J. Chung, Pullman, WA (US); Carey L. BANDARANAYAKA MUDIYANSELAGE, Pullman, WA (US)

(21) Appl. No.: 15/886,915

(22) Filed: Feb. 2, 2018

Related U.S. Application Data
(62) Division of application No. 15/210,792, filed on Jul. 14, 2016, now abandoned.

(51) Int. Cl. G01N 33/569; C07K 14/44; C12N 9/10

(52) U.S. Cl. CPC G01N 33/56905; C12Y 205/01018; C07K 14/44; G01N 2333/44; G01N 2469/20; G01N 2333/91177; C07K 2319/40; C12N 9/1088

(57) ABSTRACT

We have developed a modified indirect ELISA (MI-ELISA) using the spherical body protein-4 (SBP4) of Babesia bovis to detect antibody against diverse isolates through all infection stages in cattle. This SBP4 MI-ELISA was evaluated for sensitivity and specificity against field sera and sera from cattle infected experimentally with various doses and isolates as well as in detecting acute and persistent infection. The diagnostic specificity of the SBP4 MI-ELISA using IFA-negative sera was 100%, significantly higher than the RAP-1 cELISA (90.4%); the diagnostic sensitivity of the SBP4 MI-ELISA was 98.7% using the IFA-positive sera, in contrast to that of the RAP-1 cELISA at 60%. Results demonstrate excellent diagnostic sensitivity and specificity of the novel SBP4 MI-ELISA for cattle with acute and long-term carrier infections. Use of the SBP4 MI-ELISA assay in countries that have B. bovis-endemic herds will be pivotal in preventing the spread of this disease to non-endemic herds.

### BACKGROUND OF THE INVENTION

### Field of the Invention

This invention relates to a high throughput modified indirect enzyme-linked immunosorbent assay (MI-ELISA) developed to diagnose diverse strains of Babesia bovis in all bovine infection stages in order to effectively control intra- and inter-herd transmissions of B. bovis and to prevent the spread of B. bovis from endemic to non-endemic herds.

### Description of the Relevant Art

Bovine babesiosis, caused by protozoan parasites of the genus Babesia (order Piroplasmida, phylum Apicomplexa), is an economically important tick-borne disease, particularly in tropical and subtropical areas of the world (Bock et al. 2004. Parasitol. 129 Suppl: S247-S269; Bovine Babesiosis. 2012. In: Manual of Diagnostic Tests and Vaccines for Terrestrial Animals, 7^{th} Edition, World Organization for Animal Health (OIE), Paris). Among the various Babesia species, Babesia bovis and Babesia bigemina are widely distributed and of major importance in Africa, Asia, Australia, and Central and South America (OIE 2012, supra). Babesia species causing severe disease in naïve cattle are a potential threat to Babesia-free or non-endemic areas of the world, including the United States, given the large numbers of cattle that are moved across borders each year. The higher prevalence of B. bovis and B. bigemina in tropical and subtropical areas is closely associated with the availability of the Rhipicephalus microplus tick, the main vector of B. bovis and B. bigemina transmission to cattle and related species (Bowman and Nutall. Babesiosis of Cattle. 2008. In: Ticks: Biology, Disease and Control, Cambridge University Press, Cambridge, UK, pages 281-307). Babesia bovis infection is characterized by high fever, ataxia, anorexia, circulatory shock, and sometimes central nervous system signs due to sequestration of parasitized erythrocytes in cerebral capillaries. Babesia bovis is generally more pathogenic than other bovine Babesia species (OIE 2012, supra).

A few reports demonstrated the persistence of B. bovis in cattle infected with a moderately attenuated strain or vaccinated with a modified live vaccine (MLV) strain. Calves infected with the Mo7 strain, which is moderately attenuated by cloning and in vitro passages, were persistently infected for at least 10 months, based on PCR and other evidence (Suarez et al. 2012. Mol. Biochem. Parasitol. 185:52-57). Friesian cattle vaccinated with an attenuated strain showed evidence of persistent parasitemia for up to 47 months as determined by sub-inoculation into splenectomized calves (Pipano et al. 2002. Vet. J. 164:64-68). These two experiments, though limited, nevertheless provide strong evidence of long-term persistence of B. bovis infection in cattle. In view of this, an ideal serological assay should target a B. bovis antigen that is stably expressed for an extended period and should thus be able to detect specific antibodies throughout all stages of infection. However, to our knowledge, there is no report on long-term (>100 days post-infection or vaccination) serological monitoring of B. bovis-specific antibody responses in persistently infected cattle. One study monitored B. bovis-specific antibody responses through 98 days after challenge with a highly virulent strain, T2Bo, using the cELISA (competitive Enzyme-Linked Immunosorbent Assay) based on rhoptry-associated protein-1 (RAP-1) epitope (Goff et al. 2003. Clin. Diagn. Lab. Immunol. 10:38-43). Another study monitored antibody responses based on an immunofluorescence assay (IFA) through 60 days post-vaccination (Guglielmone et al. 1997. Vet. Parasitol. 70:33-39). In these two studies, both the cELISA and IFA were reliable for short-term monitoring of B. bovis-specific antibody responses; however, their reliability for monitoring longer post-infection periods has not yet been determined. Further defining long-term persistence of B. bovis infection and longevity of B. bovis-specific antibody responses in cattle after infection with various attenuated vaccine strains and pathogenic strains is crucial for developing control measures including higher performance diagnostics and safer vaccines against this tick-borne disease.

Several serological diagnostic assays to detect B. bovis-specific antibodies have been used as in-house assays with limited validation and quality control. The IFA test has been widely used, but low throughput and subjectivity in result interpretation are major disadvantages to its use for serological diagnosis of B. bovis infection. The complement fixation (CF) test has been used in some countries to qualify animals for importation as well as for general diagnosis (OIE 2012, supra). However, the CF test is labor-intensive, time-consuming to perform, and suffers from poor reproducibility among laboratories, perhaps attributable to inadequate standardization of both test reagents and procedure as reported in other intraerythrocytic species (Aubry and Geale. 2011. Transbound. Emerg. Dis. 58:1-30). Further, the CF test as formatted using guinea pig complement does not detect all bovine IgG antibody isotypes (Calder et al. 1996. J. Clin. Microbiol. 34:2748-2755) possibly contributing to poor diagnostic sensitivity. Due to these disadvantages, IFA and CF tests have largely been replaced by ELISAs as the sero-diagnostic test of choice for B. bovis (Bono et al. 2008. Vet. Parasitol. 157:203-210; Boonchit et al. 2004. J. Clin. Microbiol. 42:1601-1604; Goff et al. 2003, supra; Goff et al. 2006. Clin. Vaccine Immunol. 13:1212-1216; Molloy et al. 1998a. Prev. Vet. Med. 33:59-67; Molloy et al. 1998b. Parasitol. Res. 84:651-656; (OIE 2012, supra); Terkawi et al. 2011a. Clin. Vaccine Immunol. 18:337-342). Early ELISA formats for B. bovis included indirect ELISAs using whole merozoite antigen (Molloy et al. 1998a, supra) or recombinant subunit proteins (Bono et al., supra; Boonchit et al., supra). More recently, a competitive blocking ELISA (cELISA) based on an epitope of RAP-1 has been developed and evaluated with limited sera (Goff et al. 2003, supra; Goff et al. 2006, supra). Presently, there is no ELISA or other high throughput assay that has been systematically validated against diverse sera from cattle in different infection stages from various geographical areas (OIE 2012, supra). Cross-reactivity with closely-related Babesia species or other species results in relatively lower specificity for ELISAs using whole parasite antigens (Aubry and Geale, supra; Duzgun et al. 1988. Vet. Parasitol. 29:1-7). If sufficiently-conserved subunit proteins containing multiple B cell epitopes are not utilized, ELISAs using subunit proteins of B. bovis tend to have the opposite problem of relatively lower diagnostic sensitivity due to antigenic variation among B. bovis strains. To overcome the challenges in sero-diagnosis of B. bovis and to better control B. bovis infection globally, a novel, high-throughput assay having excellent diagnostic sensitivity (>95%) and excellent B. bovis species specificity (>95%) is needed. Such a serodiagnostic assay would detect antibody throughout all stages of the infection with the vast majority (ideally all) of global B. bovis isolates and would not detect antibody induced by other closely related Babesia species.

### SUMMARY OF THE INVENTION

We have developed and validated a spherical body protein-4 (SBP4) MI-ELISA to diagnose bovine species infected with antigenically diverse isolates of Babesia bovis in order to effectively control intra- and inter-herd transmissions of B. bovis and to prevent the spread of B. bovis from endemic to non-endemic herds.

In accordance with this discovery, it is an objective of the invention to provide a method of detecting acute and long-term carrier infection of diverse Babesia bovis isolates with improved sensitivity and specificity over the existing methods of detection of B. bovis in animals, resulting in a more rapid and accurate high throughput diagnosis.

It is thus an objective of this invention to provide a novel fusion protein comprising a modified Babesia bovis-specific protein (SBP4) wherein the expressed protein comprises a signal peptide-deleted SBP4 and glutathione S-transferase (GST) (GST-SBP4 antigen) fusion protein.

It is further an objective of the invention to provide an enhanced method of recombinant GST-SBP4 antigen coating and presentation using glutathione-BSA (G-BSA). This method improves the presentation quality of epitopes in the coated antigen to detect Babesia bovis-specific antibodies in test samples.

Another objective of the invention is to provide a recombinant GST-SBP4 labelled with horse-radish peroxidase (HRP) as the detection conjugate in the MI-ELISA. This novel conjugate is crucial for improved sensitivity and specificity of the MI-ELISA over the other ELISA methods in detecting B. bovis antibodies. This conjugate system allows the MI-ELISA to detect Babesia bovis antibodies in test samples from non-bovine species. This feature is not available in other indirect ELISAs based on specific-specific conjugate system.

Other objectives and advantages of this invention will become readily apparent from the ensuing description.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 depicts the contrast in sensitivity and specificity obtained in epitope presentation and detection in the MI-ELISA vs. a conventional indirect ELISA. In the MI-ELISA, the wells of the immunoassay plate are coated with G-BSA, followed by the rGST-SBP4 (signal sequence deleted) fusion protein resulting in oriented steric presentation of poly SBP4 epitopes as compared to the SBP4-coated wells of the conventional indirect ELISA. In the MI-ELISA, HRP-conjugated rGST-SBP4 fusion protein is used as the detecting reagent, thus the poly-epitope binding results in increased specificity as compared to the conventional indirect ELISA.

FIGS. 2A and 2B depict ROC (receiver operating characteristic) curve analysis to determine optimal S/N (sample OD to negative control OD) ratio cut-off for the SBP4-based MI-ELISA. Data points in FIG. 2A represent 1,254 field sera categorized as B. bovis positive (light blue) or negative (orange) by IFA. Dark blue vertical line is 3 S/N ratio cutoff for the MI-ELISA. FIG. 2B represents analysis on the area on the ROC curve with 3 S/N ratio cutoff.

FIG. 3 depicts a Western blot analysis (lanes A-J) of cattle sera positive by the RAP-1-based cELISA but negative by the SBP4-based MI-ELISA and IFA. Lanes: A. Molecular weight marker, B. K42-#21, C. W31-#Y-3, D. W31-#Y-11, E. W31-#0-3, F. W31-#Y-9, G. W31-#0-9, H. W31-#Y-10, I. W31-#Y-15, J. P21-#224, K. Positive control serum with a band at 75 kd representing B. bovis RAP-1 protein, J. Negative control serum.

FIG. 4 depicts long-term monitoring of Babesia bovis parasitemia in blood from calves challenged with a high dose of the T2Bo or Mo7 strain. DPI represents days post-inoculation, with the red dots depicting positive parasitemia days of calf 41466 (infected with the T2Bo strain of B. bovis) and purple dots, the positive parasitemia days of calf 41441 (infected with Mo9). Parasitemia was monitored by nested PCR on a daily basis for the first fifty DPI and on a biweekly basis thereafter until 10 months post-inoculation.

FIGS. 5A, 5B, 5C, and 5D depict long-term monitoring of Babesia bovis antibody responses by ELISA in serum specimens from calves challenged with a high dose of the T2Bo or Mo7 strain. S/N ratio indicates sample optical density (OD) to negative control OD ratio for the SBP4-based MI-ELISA and % I indicates % inhibition for the RAP-1 cELISA. Red horizontal lines are 3 S/N ratio cutoffs for the MI-ELISA or 21% inhibition cutoff for the RAP-1 cELISA, respectively. The calf infected with a high dose of moderately attenuated Mo7 strain had detectable antibody beginning at 10, 11, and 14 days DPI by the SBP4 MI-ELISA (FIG. 5A) and the RAP-1 cELISA (FIG. 5B). The calf infected with a high dose of T2Bo strain had detectable antibody beginning at 13, 14, and 15 DPI by the SBP4 MI-ELISA (FIG. 5C) and the RAP-1 cELISA (FIG. 5D).

FIGS. 6A, 6B, 6C, and 6D depict long-term monitoring of B. bovis antibody responses by IFA in serum specimens from calves challenged with a high dose of the T2Bo or Mo7 strain. DPI represents days post-inoculation. A positive IFA result was defined as fluorescence ≥1+. The calf infected with a high dose of moderately attenuated Mo7 strain had detectable antibody beginning at 10, 11, and 14 days DPI by IFA (FIGS. 6A and 6B). The calf infected with a high dose of T2Bo strain had detectable antibody beginning at 13, 14, and 15 DPI by IFA (FIGS. 6C and 6D).

FIG. 7 depicts long-term monitoring of B. bovis parasitemia in blood from calves challenged with a low dose of the Tf197-4 or Mo7 strain. DPI represents days post-inoculation, with the red and pink dots depicting positive parasitemia days of calves 1292 and 1290 (infected with the Tf197-4 strain of B. bovis), respectively and green and blue dots, the positive parasitemia days of calves 1291 and 1287 (infected with Mo7), respectively. Parasitemia was monitored by nested PCR on a daily basis for the first twenty DPI and on a biweekly basis thereafter until 11 months post-inoculation.

FIGS. 8A, 8B, 8C, and 8D depict long-term monitoring of B. bovis antibody responses by the SBP4-based MI-ELISA in serum specimens from calves challenged with a low dose of the Tf-137-4 or Mo7 strain. DPI represents days post-inoculation. S/N ratio indicates sample optical density (OD) to negative control OD ratio for the SBP4-based MI-ELISA. Red horizontal line is 3 S/N ratio cutoff for the MI-ELISA. FIGS. 8A and 8C depict calves infected with a low dose of moderately attenuated Mo7 strain; FIGS. 8B and 8D depict calves infected with a low dose of Tf-137-4 strain.

FIGS. 9A, 9B, 9C, and 9D depict long-term monitoring of B. bovis antibody responses by the RAP-1-based cELISA in serum specimens from calves challenged with the low dose of the Tf-137-4 or Mo7 strain. DPI represents days post-inoculation. % I indicates % inhibition for the RAP-1 cELISA. Red horizontal line is 21% inhibition cutoff for the RAP-1 cELISA. FIGS. 9A and 9C depict calves infected with a low dose of moderately attenuated Mo7 strain; FIGS. 9B and 9D depict calves infected with a low dose of Tf-137-4 strain.

FIGS. 10A, 10B, 10C, and 10D depict long-term monitoring of B. bovis antibody responses by IFA in serum specimens from calves challenged with a low dose of the Tf-137-4 or Mo7 strain. DPI represents days post-inoculation. A positive IFA result was defined as fluorescence ≥21+. FIGS. 10A and 10C depict calves infected with a low dose of moderately attenuated Mo7 strain; FIGS. 10B and 10D depict calves infected with a low dose of Tf-137-4 strain.

### DETAILED DESCRIPTION OF THE INVENTION

Most cattle survive acute B. bovis infection, and some studies have demonstrated that recovered cattle become clinically inapparent carriers that could serve as reservoirs for intra- and inter-herd transmission (Bock et al., supra; Calder et al., supra; Goff et al. 2003, supra; Suarez et al. 2012, supra). However, long-term kinetics and durations of B. bovis parasitemia and antibody responses remain to be defined. Defining these aspects in cattle infected with diverse B. bovis strains in variable doses is crucial for establishing effective control measures against transmission of this important trans-boundary disease, particularly from endemic to non-endemic and Babesia-free countries.

A typical characteristic of B. bovis, but not of B. bigemina, is the ability to escape the immune system of the host using rapid antigenic variation, cytoadhesion and capillary sequestration, and binding of host proteins to the surface of infected red blood cells (Allred, D. R. 2003. Trends. Parasitol. 19:51-55). It is believed that these mechanisms contribute to the establishment of persistent infections, which usually result in the development of long term and fluctuating immune responses. Therefore, if persistent infections are characterized frequently by very low levels of circulating antibodies and often undetectable parasitemia, then detection of persistently infected animals or unknown infected carriers may require exquisitely sensitive methods of detection.

A recent comparative study with five subunit proteins of B. bovis in the conventional indirect ELISA format reported that spherical body protein-4 (SBP4) was relatively better than MSA-2c (merozoite surface antigen-2c), RAP-1/CT (rhoptry-associated protein-1/carboxy terminal), TRAP-T thrombospondin-related anonymous protein-truncated) and SBP1 (spherical body protein-1) in both diagnostic sensitivity and specificity (Terkawi et al. 2011a, supra). Further study defined that SBP4 was produced and secreted in a stable and dominant manner through all stages of the B. bovis life cycle (Terkawi et al. 2011 b, Mol. Biochem. Parasitol. 178:40-45). However, only 85% diagnostic concordance (84.5% sensitivity and 86.2% specificity) was observed between an SBP4-based conventional indirect ELISA and IFA with 469 sera collected from five countries suggesting the need for further improvement (Terkawi et al. 2011a, supra). Our main objectives are to 1) develop and evaluate a novel SBP4 MI-ELISA format using recombinant SBP4 as the coating antigen and detection conjugate to produce higher diagnostic performance than previously reported antibody assays, 2) compare the performance of this novel SBP4 MI-ELISA to the reference IFA and to the previously developed RAP-1 cELISA using cattle sera from non-endemic, endemic and epidemic areas, and 3) demonstrate that the novel SBP4 MI-ELISA would detect antibody in sera from cattle that had long term infections documented by at least occasional nested PCR positive tests and then compare these results with those from IFA and RAP-1 cELISA.

A well-defined, reliable target antigen and robust assay format are critical to the development of a diagnostic assay with high specificity and sensitivity against sera from long-term unapparent carriers. Several sero-diagnostic assays including the CF test (Mahoney, D. F. 1962. Aust. Vet. J. 38:48-52), the indirect hemagglutination (Goodger, B. F. 1971. Aust. Vet. J. 47:251-256), the rapid card agglutination test (Todoric and Kuttler. 1974. Am. J. Vet. Res. 35:1347-1350), the immunofluorescence assay (IFA, Goff et al. 1982. Vet. Parasitol. 11:109-120) and the cELISA (Goff et al. 2003, supra) have been developed, and some have been used extensively in diagnostic labs. Although most of these assays provide diagnostic performance appropriate for herd monitoring that requires moderate diagnostic accuracy, they are not sufficient for disease and disease-free certification of individual animals. Currently the CF test, IFA and ELISA are OIE-alternative tests for international trade of bovines. However, whether these OIE-alternative assays are acceptable for uses in disease or disease-free certification requiring high diagnostic specificity and sensitivity has not been validated.

A cELISA using an epitope of RAP-1 has been developed and tested with limited sera (Goff et al. 2003, supra; Goff et al. 2006, supra). By their very design, single epitope-based cELISAs are susceptible to low diagnostic sensitivity when used for infectious agents with high antigenic variation such as B. bovis. Conventional indirect ELISAs using well-characterized subunit proteins or well-purified whole parasites may have an advantage in diagnostic sensitivity due to leveraging far more B cell epitopes than cELISAs. Nevertheless, antigens for both cELISAs and indirect ELISAs must be carefully selected for species-limited conservation to avoid cross reactivity against other Babesia or related species that could have a deleterious effect on specificity. A recently published comparative analysis of several B. bovis proteins in the conventional indirect ELISA format suggested enormous variations in diagnostic performance depending on subunit proteins. This study also indicated relatively better diagnostic performance of the indirect ELISA based on SBP4 (85% agreement with IFA results) than other B. bovis proteins when tested against sera from five different countries (Terkawi et al. 2011a, supra). A moderate (85%) diagnostic agreement of the SBP4-based conventional indirect ELISA against 469 sera validated in an IFA test in this comparative study indicates the need for further improvement and optimization in format and other variables followed by extensive validation using more diverse sera.

To this end, in the present study, the SBP4-based modified indirect ELISA, SBP4-MI-ELISA, was developed using the recombinant fusion protein rGST-SBP4. The rGST-SBP4 fusion protein (SEQ ID NO: 1) is comprised of glutathione S-transferase (GST) and recombinant spherical body protein-4 modified by having the signal sequence deleted. The rGST-SBP4 fusion protein is used as the coating antigen in the SBP4-MI-ELISA and also as the detection antigen (rGST-SBP4-HRP) when conjugated with horseradish peroxidase. The cutoff for positive or negative antibody detection used in this assay was 3.0 S/N ratio which gave an excellent combination of 100% specificity and 97.6% sensitivity when tested with diverse sera collected from endemic, epidemic and non-endemic areas. Surprisingly, the SBP4-based MI-ELISA had a significantly better diagnostic performance than results reported for the indirect ELISA based on SBP4 (Terkawi et al. 2011a, supra) and the cELISA based on RAP-1 (Goff et al. 2003, supra; Goff et al. 2006, supra) in several aspects. First, the SBP4-based MI-ELISA detected B. bovis antibody earlier after infection than IFA (˜1.5 day delayed positive detection) and the RAP-1-based cELISA (˜9.5 day delayed positive detection). Second, the MI-ELISA was more reliable for long term monitoring of antibody responses in the carrier stage of B. bovis infection than the cELISA, which was unreliable after 5-10 months post-infection when parasitemia was still detectable by PCR. Third, the MI-ELISA had 37% and 9.2% higher diagnostic sensitivity than the cELISA against sera collected from B. bovis-endemic regions in Mexico and Australian herds, respectively. Fourth, diagnostic specificity (100%) of the MI-ELISA was comparable to the cELISA when tested against sera from northwestern U.S. herds kept in tick-free barns. In addition, the MI-ELISA had superior diagnostic specificity to the cELISA against sera from the southern U.S. (100% versus 90.4%) and Mexico (100% versus 93.8%). Fifth, the MI-ELISA had no positive cross-reactivity with sera from cattle infected with B. bigemina or Anaplasma marginale. Persistence of antibody responses specific for B. bovis SBP4 even at DPIs with no detectable parasitemia by nested PCR clearly indicates the relative advantage of certain antibody-based diagnostics over parasitemia detection assays for this disease. This of course depends on the availability of an accurate, high-throughput serology assay with more objective interpretation than IFA, such as the SBP4 MI-ELISA described here. This assay is a more reliable tool for identifying animals in the carrier stage of B. bovis infection than PCR-based parasitemia detection or antibody detection by the RAP-1 cELISA.

It is understood that the present invention also encompasses rGST-SBP4 variants. A preferred rGST-SBP4 variant is one having at least 90% amino acid sequence similarity to the rGST-SBP4 amino acid sequence (SEQ ID NO: 1), a more preferred rGST-SBP4 variant is one having at least 95% amino acid sequence similarity to SEQ ID NO: 1 and a most preferred rGST-SBP4 variant is one having at least 99% amino acid sequence similarity to SEQ ID NO: 1 as defined by the algorithm, CLUSTRAL or PILEUP.

A “variant” of rGST-SBP4 may have an amino acid sequence that is different by one or more amino acid “substitutions”. The variant may have “conservative substitutions”, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions, or both. Guidance in determining which and how many amino acid residues may be substituted, inserted or deleted without abolishing biological or immunological activity may be found using computer programs well known in the art, for example, DNASTAR software. The term “biological activity” refers to rGST-SBP4 having structural, regulatory or biochemical functions of the naturally occurring protein. Likewise, “immunological activity” defines the capability of the natural, recombinant or synthetic rGST-SBP4, or any oligopeptide thereof, to induce a specific immune response in appropriate animals or cells and to bind with specific antibodies.

The phrase “conservative substitution” also includes the use of a chemically derivatized residue in place of a non-derivatized residue. “Chemical derivative” refers to the chemical modification of a nucleic acid sequence encoding rGST-SBP4 or the encoded rGST-SBP4 wherein the subject nucleic acid or polypeptide has one or more residues chemically derivatized by reaction of a functional side group. Examples of such modifications would be replacement of hydrogen by an alkyl, acyl, or amino group; however, replacements are not limited to these groups. A nucleic acid derivative would encode a polypeptide which retains essential biological characteristics of natural rGST-SBP4. Also included are those peptides which contain one or more naturally-occurring amino acid derivatives of the twenty standard amino acids, e.g., 5-hydroxylysine or ornithine may be substituted for lysine.

The term “peptide” as used herein refers to a molecular chain of amino acids with a biological activity (e.g., capable of binding antibody specific for B. bovis), and does not refer to a specific length of the product. Thus, inter alia, proteins, oligopeptides, polypeptides and fusion proteins as well as fusion peptides are included. Further, GST-SBP4+ and rGST-SBP4 are interchangeable as reagents for detecting

B. bovis-specific antibodies, for generating B. bovis-specific antibodies, and for vaccine development. Thus, inter alia, reference to GST-SBP4+ encompasses rGST-SBP4, and reference to rGST-SBP4 encompasses GST-SBP4+.

The term “antibody,” as used herein, includes, but is not limited to a polypeptide substantially encoded by an immunoglobulin gene or immunoglobulin genes, or fragments thereof which specifically bind and recognize an analyte (antigen). The phrases “specifically binds to” or “specifically immunoreactive with”, when referring to an antibody or other binding moiety refers to a binding reaction which is determinative of the presence of the target analyte in the presence of a heterogeneous population of proteins and other biologics. Thus, under designated assay conditions, the specified binding moieties bind preferentially to a particular target analyte and do not bind in a significant amount to other components present in a test sample. Specific binding to a target analyte under such conditions may require a binding moiety that is selected for its specificity for a particular target analyte. A variety of immunoassay formats may be used to select antibodies specifically immunoreactive with a particular protein. For example, solid-phase ELISA immunoassays are routinely used to select monoclonal antibodies specifically immunoreactive with an analyte. See Harlow and Lane (1988), Antibodies, A Laboratory Manual, Cold Spring Harbor Publications, New York, for a description of immunoassay formats and conditions that can be used to determine specific immuno-reactivity. Typically a specific or selective reaction will be at least twice background signal to noise and more typically more-than 10 to 100 times background.

The DNA sequences of the invention can be used to prepare recombinant DNA molecules by cloning in any suitable vector. A variety of vector-host combinations may be employed in practicing the present invention. Host cells may be either prokaryotic or eukaryotic, and, when the host cells are bacterial cells, they may be either gram-negative or grain-positive bacteria. Without being limited thereto, examples of hosts suitable for use herein are prokaryotic and eukaryotic hosts such as E. coli K12 or XL1 Blue cells and related bacteria, Saccharomyces cerevisiae, Sf9 or Sf21 insect cells (Spodoptera frugiperda), Chinese hamster ovary cells, and plant cells in culture. However, other hosts may also be utilized.

Vectors used in practicing the present invention are selected to be operable as cloning vectors or expression vectors in the selected host cell. Numerous vectors are known to those of skill in the art, and selection of an appropriate vector and host cell is a matter of choice. This invention encompasses a hybrid vector, that comprises a vector capable of replication, transcription and expression of DNA segments operably coupled thereto; and a DNA segment encoding a polypeptide of this invention comprising the peptide disclosed herein operatively coupled thereto, wherein when the vector is placed in an appropriate host it can express the polypeptide encoded by the DNA segment. Examples of such vectors are pGex (Pharmacia), baculovirus, pET-9d (Novagen), pRSET T7 (Invitrogen), pTriplEx2 plasmid vector and pMal-c2 plasmid vector (Invitrogen). The vector may be a eukaryotic or a prokaryotic vector depending on the host selected for transfection and in which the gene product is going to be expressed.

Still part of this invention is another hybrid vector, that comprises a vector capable of replication, transcription and expression of DNA segments operably coupled thereto; and a DNA segment comprising a DNA fragment encoding at least one of the polypeptides of the invention and a second unrelated DNA segment, both sequences being operably coupled to one another and to the vector. The preparation of the hybrid vector described above is known in the art and need not be further described herein (Smith, D., and K. Johnson, “Single Step Purification of Polypeptides Expressed in E. coli as Fusions with Glutathione S-transferase”, Gene, 67:31(1988); Studier, F. W., et al., “Use of T7 RNA Polymerase to Direct Expression of Cloned Genes”, Meth. Enzymol., 185:60-89 (1990)).

The antigenic peptides of the invention are produced by growing host cells transformed by the expression vectors described above under conditions whereby the antigen is produced. The antigens are then isolated from the host cells.

Also an important part of this invention is a method of diagnosing B. bovis infection that comprises contacting a body substance with rGST-SBP4 of this invention; and detecting any selective binding of the rGST-SBP4 to any anti-B. bovis antibodies in a body substance using the MI-ELISA of the invention. The present antibody-polypeptide binding complex may be detected by a variety of methods such as is described above. Examples of body fluids are blood, serum, saliva, urine, and the like, including aqueous humor, vitreous humor, blood plasma, cerebrospinal fluid, perilymph, endolymph, lymph, mucus, pericardial fluid, pleural fluid, synovial fluid, milk, colostrum, or oral fluids. Methods for the preparation of the body fluid are standard in the art and need not be further detailed herein (see, for example, Manual of Clinical Microbiology, Chapter 8, “Collection, Handling and Processing of Specimens”, 4th edition, Eds, Lennette, E. H., et al., American Society for Microbiology (1986)).

Still part of this invention is a kit for the diagnosis of B. bovis infection, that comprises the rGST-SBP4 peptide of this invention; and instructions for use of the kit. This kit may be utilized for the detection of endogenous antibodies produced by a subject that is afflicted with babesiosis. Even at the early stages where the parasite is commencing invasion of a subject's cells, some amount of B. bovis-specific antibody may be detected in serum. In addition to the above, the kits may also comprise a control, anti-antibodies, protein A/G, and the like, suitable for conducting the different assays referred to above.

In another aspect of the invention, immunoassays using the disclosed rGST-SBP4 peptide are provided. In the context of this aspect of the invention, the rGST-SBP4 peptide that has specificity for the antibodies in the biological sample is bound to a substrate. The term “bound” refers to both covalent and non-covalent attachment of a peptide to a substrate. Thus, polypeptides can be covalently bound to the substrate via a linker physically attached to a substrate or non-covalently bound to a substrate (e.g., adsorbed to a substrate surface, for example, a polystyrene surface). In various embodiments, the substrate can be selected from tubes, cylinders, beads, discs, silicon chips, microplates, nitrocellulose membrane, nylon membrane, porous membranes, non-porous membranes, plastic, polymer, silicon, polymeric pins, a plurality of microtiter wells, or any combinations thereof. The composition of the substrate can also be varied. For example, substrates (alternatively referred to as a support) can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, polysulfone, or the like. Thus, the substrate may be any surface or support upon which a peptide, such as the rGST-SBP4 peptide, can be immobilized, including one or more of a solid support (e.g., glass such as a glass slide or a coated plate, silica, plastic or derivatized plastic, paramagnetic or non-magnetic metal), a semi-solid support (e.g., a polymeric material, a gel, agarose, or other matrix), and/or a porous support (e.g., a filter, a nylon or nitrocellulose membrane or other membrane). In some embodiments, synthetic polymers can be used as a substrate, including, e.g., polystyrene, polypropylene, polyglycidylmethacrylate, aminated or carboxylated polystyrenes, polyacrylamides, polyamides, polyvinylchlorides, and the like. In preferred embodiments, the substrate comprises a microtiter immunoassay plate or other surface suitable for use in an ELISA.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

### EXAMPLES

Having now generally described this invention, the same will be better understood by reference to certain specific examples, which are included herein only to further illustrate the invention and are not intended to limit the scope of the invention as defined by the claims.

### Example 1

### Cloning and Expression of Spherical Body Protein-4

A cDNA encoding the recombinant fusion protein rGST-SBP4 comprised of glutathione S-transferase (GST) and recombinant spherical body protein-4 (SBP4) of B. bovis (T2Bo strain) modified by having the signal sequence deleted (GenBank accession number: KX524469) was cloned into the pGEX-2T vector (New England BioLabs, Ipswich, Mass., USA). This recombinant (r) GST-SBP4-containing vector was transformed to BL21 cells (New England BioLabs, Ipswich, Mass., USA) and expressed as follows. Briefly, 200 mL of an overnight culture of pGEX-2T/SBP4-transformed BL21 cells were inoculated into 1.8 liters of LB medium (Becton, Dickinson and Company, Sparks, Md., USA) containing 0.01% ampicillin and grown at 37° C. for 3 hr. Following addition of 0.048 g of isopropyl-β-D-thiogalacto-pyranoside (IPTG; US Biologicals, Salem, Mass., USA), the bacteria were incubated at 37° C. for an additional 5 hr. The bacteria were harvested by centrifugation at 4.5×10³ g for 25 min and then resuspended in phosphate buffered saline. The resuspended pellet was sonicated on ice and Triton X-100 (Sigma-Aldrich, St. Louis, Mo., USA) was added to a final concentration of 1%. The sonicated suspension was centrifuged at 5.7×10⁴ g for 30 min. Finally, the clarified supernatant containing the rGST-SBP4 fusion protein was collected and stored at −80° C. until used for antigen coating or further purification to make the horseradish peroxidase (HRP)-conjugated recombinant SBP4.

### Example 2

### Purification of rGST-SBP4 Fusion Protein; Conjugation with Horseradish Peroxidase

The recombinant GST-SBP4 fusion protein was purified from the supernatant described above using glutathione-agarose beads (Sigma G4510) according to the manufacturer's instruction. Briefly, glutathione-agarose beads equilibrated with PBS containing 1% Triton-X 100 were resuspended in the clarified lysate to agitate for 60 minutes at room temperature. The beads capturing recombinant GST-SBP4 were pelleted by centrifugation and washed with PBS three times. After the last wash of the beads, the recombinant GST-SBP4 was eluted from the beads using the elution buffer containing 30 mM reduced Glutathione in 50 mM Tris-HCl (pH 9.0). The eluted rGST-SBP4 was conjugated with horse-radish peroxidase according to the method previously described (Nakane and Kawaoi. 1974. J. Histochem. Cytochem. 22:1084-1091). The conjugate concentrate was stabilized by adding a final concentration of 10% heat-inactivated goat serum and stored at 2-7° C.

### Example 3

### Immunofluorescence Antibody Assay

The IFA was performed as previously described (Goff et al. 2006, supra; Goff et al. 1982, supra) using 50 μl of a 1/50 dilution of serum in serum dilution buffer (1×PBS) and substrate slides prepared using red blood cells parasitized by two B. bovis strains Mo7 and T2Bo. A positive result was defined as fluorescence equal (1+) to or greater than (2 to 3+) that of a weak positive control sample defined by IFA, western blot and RAP-1 cELISA after collection from a bovine experimentally-infected with Mo7 stain. A negative result was defined as comparable to the background fluorescence of a negative control serum collected from a B. bovis-negative herd in the northwestern U.S.

### Example 4

### Western Blot Analysis for Detection of Anti-RAP-1 Antibody in Bovine Sera

Western blot analysis to detect B. bovis-specific antibodies directed against RAP-1 in bovine sera was performed using recombinant RAP-1 (rRAP-1) according to the previously described method (Nakane and Kawaoi, supra) with some modifications. Briefly, rRAP-1 was boiled for three minutes in sample buffer (DGel Sciences, Montreal, Canada) and separated by electrophoresis using sodium dodecyl sulfate-polyacrylamide gel (Bio-Rad, Hercules, Calif., USA). Transfer to nitrocellulose was performed by standard techniques (Chung et al. 2014. J. Vet. Diagn. Invest. 26:61-71) and membranes were blocked in tris-tween-20 buffer containing 5% skim milk. Bovine serum antibody bound to the rRAP-1 band was detected with HRP-conjugated goat anti-bovine IgG (KPL, Gaithersburg, Md., USA) (Terkawi et al. 2011a, b, supra).

### Example 5

### Bovine Sera Used for Assay Evaluations

Negative sera were collected from 302 uninfected cattle in northwestern US herds that were maintained in barns free of B. bovis-transmitting ticks and that had no history of clinical babesiosis. Ninety-four sera were collected from Texas herds with unknown B. bovis status, 93 of these sera were negative in the indirect immunofluorescence assay (IFA). Thirty-two sera, negative by IFA, were selected as additional negative samples from B. bovis endemic areas. These 427 negative sera were used to evaluate the diagnostic specificity of the RAP-1-based cELISA and the newly developed rSBP4-based MI-ELISA.

B. bovis-positive sera (n=826) were obtained from cattle with positive results by IFA, and were used to evaluate the diagnostic sensitivity of the MI-ELISA and the RAP-1 cELISA. Two hundred twenty-eight of these 826 sera were from herds in Australia vaccinated with an attenuated B. bovis Dixie strain (n=180) or challenged with virulent W strain (n=48) in Australia, and the remaining 598 sera were collected from several areas in Mexico. Four hundred and two additional IFA-positive sera were selected from sequential serum collections from six experimentally-infected cattle and used to further evaluate relative sensitivity of the MI-ELISA and the RAP-1 cELISA. Excluding the 32 negative samples at early post-infection days and 402 positive samples from experimentally-infected animals, a total of 1,254 field serum samples were used to evaluate likely diagnostic sensitivity and specificity resulting in a statistical power of 95% confidence within a 2% margin of error.

### Example 6

### Modified Indirect ELISA

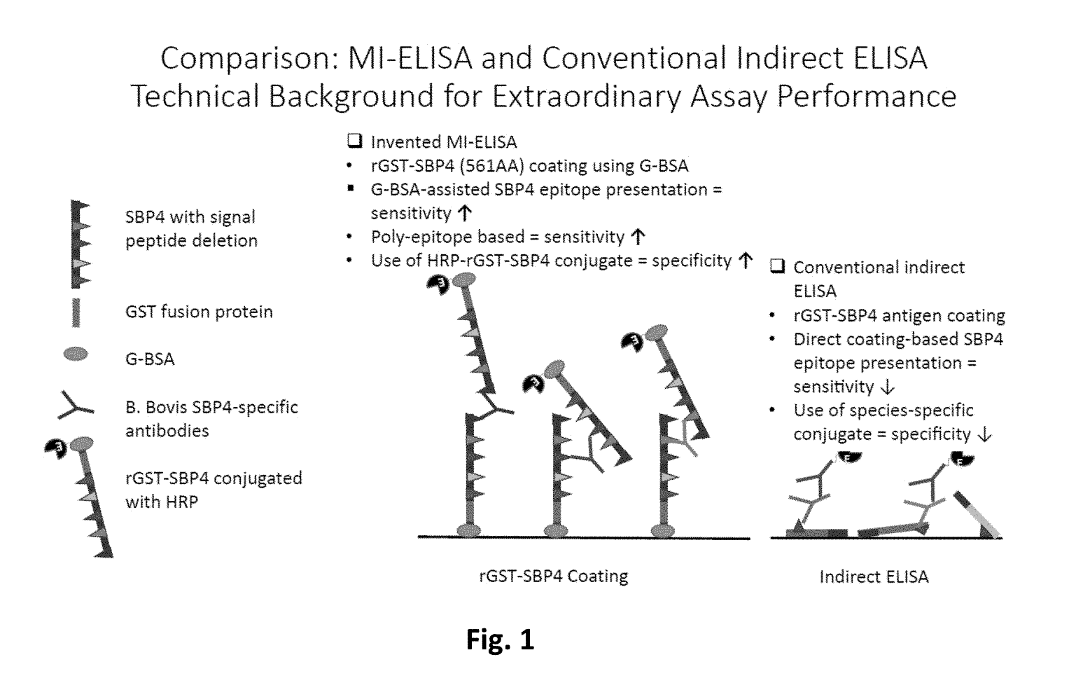

Two main features of the rGST-SBP4-based MI-ELISA for highly specific and sensitive diagnosis of B. bovis infection in cattle include 1) glutathione-BSA catcher-based purification/coating of rGST-SBP4 as antibody capture antigen and use of HRP-conjugated rGST-SBP4 as detection system (FIG. 1). Glutathione in G-BSA catcher selectively binds to GST without blocking the antibody binding epitopes in SBP4 sequence, resulting in high purity coating of rGST-SBP4 and optimal and standardized steric presentation of antibody epitopes in SBP4 to antibodies. Use of HRP-conjugated rGST-SBP4 reduce the reactivity to non-specific antibodies, contributing to enhanced specificity and sensitivity of the MI-ELISA when compared to conventional indirect ELISA using HRP conjugate made with species-specific immunoglobulins as detection system. The rGST-SBP4-based MI-ELISA was prepared as follows. Immunoassay plates (96 wells; Costar, Vernon Hills, Ill., USA) were treated with 0.08 μg/well of glutathione-bovine serum albumin, incubated overnight at 4° C., and then incubated for 2 hours at 37° C. with 200 μl/well of blocking buffer (0.05M potassium phosphate buffer containing 0.5% bovine serum albumin). Subsequently, the blocking buffer was removed, and the plates were dried overnight. Fifty microliters of a dilution of the recombinant fusion protein antigen rGST-SBP4 that gave approximately 0.08 optical density (OD) at 450 nm (A450) when tested against the negative reference serum was added to the prepared 96-well plates. Finally, the antigen-coated plates were stored individually in polyester film bags (IMPAK Co. Los Angeles, Calif., USA) at 4° C. until used.

For the SBP4 MI-ELISA testing procedure, 50 μl/well of test serum were added to wells of the plate and incubated at room temperature for 30 minutes. Wells were then washed three times with 250 μl of wash buffer (PBS+0.05% Tween 20) per well. Fifty μl of HRP-conjugated rGST-SBP4 diluted in conjugate diluting buffer (PBS containing 1% bovine serum albumin) were added to each well of the plate, and the plates were incubated at room temperature for 30 min. After wells were again washed three times with 250 μl of wash buffer per well, 50 μl of tetramethylbenzidine substrate (SurModics, Eden Prairie, Minn., USA) were added to each well, and the plates were incubated at room temperature for 15 min. The reactions were then stopped with 50 μl of 1.5% sodium fluoride solution (SurModics, Eden Prairie, Minn., USA) per well. The optical densities (OD) of the assay wells were read at 450 nm (OD₄₅₀) with a microplate spectrophotometer and results were calculated as the ratio of sample OD to negative control OD (S/N). Specifically, the OD₄₅₀ of the sample in question was divided by the OD₄₅₀ of a negative control analyzed in the same assay run.

The SBP4 MI-ELISA was optimized by evaluation of several format variables including the antigen coating protocol, serum incubation time, wash buffer, and concentration of HRP/GST-SBP4 conjugate used to detect antibody binding. Optimization utilized a small set of 6 samples that included two known negative sera and four two-fold dilutions of a B. bovis-positive serum in negative serum ranging above and below the end-point detection (Table 1). The optimized format included the use of 50 μl undiluted serum to maximize assay simplicity.

TABLE 1
Calibration of the SBP4 MI-ELISA with reference sera

| Sample, dilution | OD | | Mean OD | S/N | Result |
|---|---|---|---|---|---|
| Positive serum C167, | 0.683 | 0.675 | 0.679 | 7.5 | + |
| 1/64 | | | | | |
| Positive serum C167, | 0.395 | 0.392 | 0.394 | 4.3 | + |
| 1/128 | | | | | |
| Positive serum C167, | 0.287 | 0.269 | 0.278 | 3.1 | + |
| 1/256 | | | | | |
| Positive serum C167, | 0.194 | 0.180 | 0.187 | 2.1 | − |
| 1/512 | | | | | |
| Negative serum #37 | 0.133 | 0.112 | 0.123 | 1.3 | − |
| Negative serum #312 | 0.155 | 0.142 | 0.149 | 1.6 | − |

Positive ≥3 sample optical density (OD) to negative control OD ratio

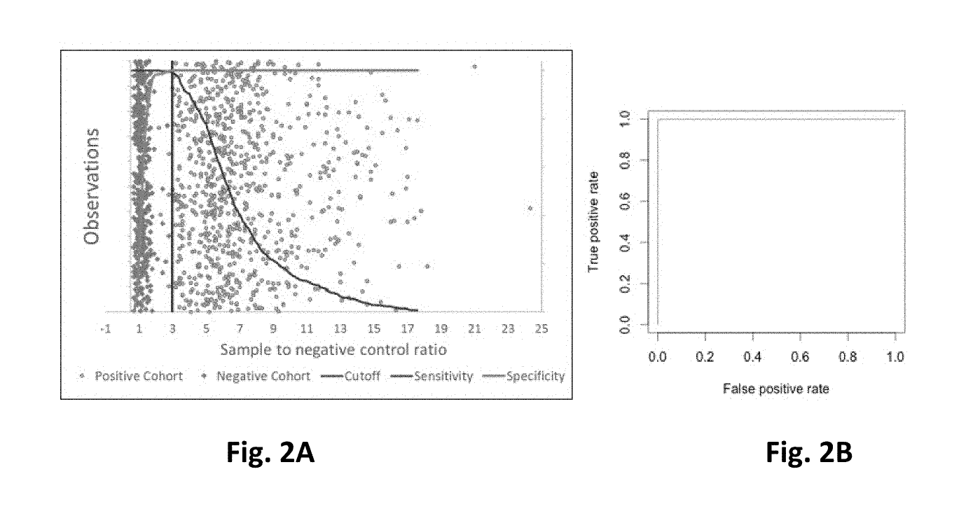

Using this optimized SBP4 MI-ELISA format, S/N ratios were determined for 1,254 sera that were already categorized as either B. bovis-IFA positive or -IFA negative. Scatter plot and ROC curve analysis were carried out using the S/N ratio data and IFA categorization (FIG. 2). This analysis resulted in a maximum value for the combined sensitivity and specificity at a cutoff of approximately 2.0 (99.6% sensitivity, 98.4% specificity). The least difference between sensitivity and specificity occurred at a cutoff of approximately 2.4 (99.0% sensitivity, 98.6% specificity). The minimum cutoff that gave 100% sensitivity was 1.5, but this was at the expense of specificity dropping to 83.6%. The maximum cutoff that gave 100% specificity was 2.9 which resulted in 97.6% sensitivity. Using a more convenient cutoff of ≥3.0 yielded sensitivity and specificity values essentially identical to a cutoff of ≥2.9. The area under the ROC curve (AUC) with this cutoff was 0.9987, which is close to the perfect classification value of 1.00, indicating high accuracy of the 3.0 S/N ratio cutoff for classifying serum samples into positive or negative. Thus, a SBP4 MI-ELISA cutoff of ≥3 was selected for further use to provide maximum sensitivity with very good specificity (FIG. 2).

To compare the specificity of the SBP4 MI-ELISA and the RAP-1 cELISA, 302 sera collected from northwestern U.S. herds maintained in barns free of B. bovis tick vectors and negative by IFA using two different B. bovis strains as IFA substrate were tested. All 302 sera were negative using both ELISA assays, generating no false positives and a resultant diagnostic specificity of 100% for both assays (Table 2). Furthermore, the SBP4 MI-ELISA had 100% diagnostic specificity when evaluated with 32 IFA-negative sera collected from B. bovis-endemic regions of Mexico (Table 2). However, the RAP-1 cELISA had a lower diagnostic specificity of 93.8% as two of the same 32 IFA-negative sera were positive (Table 2).

TABLE 2
Diagnostic Specificity of SBP4 MI-ELISA and RAP-1 cELISA

| Diagnostic Specificity | | | | |
|---|---|---|---|---|
| against | SBP4 MI-ELISA | | RAP-1 cELISA | |
| Negative sera collected | 100% | (302−/302*) | 100% | (302−/302) |
| from northwestern United | | | | |
| States | | | | |
| Negative sera collected | 100% | (32−/32*) | 93.8% | (30−/32) |
| from endemic regions of | | | | |
| Mexico | | | | |
| Negative sera collected | 0/94 | (0%) | 9/94 | (9.6%) |
| from Texas | | | | |

*Negative sera defined by IFA-negative results. Cut-offs for positive result were 3 S/N (sample optical density to negative control optical density) ratio for SBP4 MI-ELISA and 21% inhibition for RAP-1 cELISA.

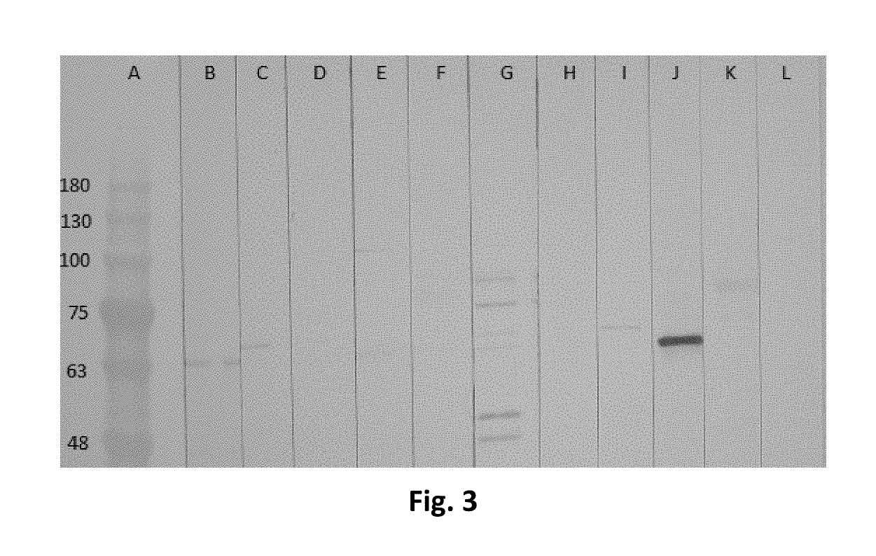

Ninety-three of 94 additional sera collected from Texas herds as potential negative samples were negative by IFA, with the remaining sample being weakly IFA positive. Nine of the 93 IFA negative sera were positive by RAP-1 cELISA (90.3% specificity) while none were positive by SBP4 MI-ELISA (100% specificity)(Table 2). All nine IFA-negative sera with discrepant results between the RAP-1 cELISA and the SBP4 MI-ELISA were negative in RAP-1 western blot analysis (FIG. 3), supporting the possibility of false positive RAP-1 cELISA results with these nine sera. Interestingly, the one weak IFA positive serum from the 94 Texas samples was negative by RAP-1 cELISA and SBP4 MI-ELISA, introducing the possibility that this serum was IFA false positive, although this was not proven. Ultimately, the SBP4 MI-ELISA had significantly (p-value=0.006) better diagnostic specificity than the RAP-1 cELISA against sera from Texas using IFA as the reference assay.

To evaluate the sensitivity of the SBP4 MI-ELISA and the RAP-1 cELISA against well-characterized positive sera, 402 IFA-positive sera collected at various DPIs from six cattle experimentally infected with one of three different B. bovis strains were analyzed. Of the 402 sera, 335 sera were strong IFA positives while the remaining 67 sera were weak IFA positives. The RAP-1 cELISA had a sensitivity of 84.6%, with 340 of the 402 IFA positive sera being RAP-1 cELISA positive (Table 3). The SBP4 MI-ELISA had a sensitivity of 100% as all 402 IFA positive sera were positive. For these experimentally infected calves, the SBP4 MI-ELISA had significantly (p<0.001) better sensitivity than the RAP-1 cELISA.

TABLE 3
Diagnostic Sensitivity of the SBP4 MI-ELISA and the RAP-1 cELISA

| Diagnostic Sensitivity against | SBP4 MI-ELISA | RAP-1 cELISA |
|---|---|---|
| IFA-positive serum samples collected | 100% (402+/402) | 84.6% |
| from six experimentally-infected | | (340+/402) |
| calves | | |
| IFA-positive sera collected from | 95.2% (217+/228) | 86.0% |
| herds vaccinated and/or challenged | | (196+/228) |
| with virulent W strain in Australia | | |
| IFA-positive serum samples collected | 98.7% (590+/598) | 60.0% |
| from B. bovis-endemic regions of | | (359+/598 |
| Mexico | | |

Cut-offs for positive result were 3 S/N (sample optical density to negative control optical density) ratio for SBP4 MI-ELISA, 21% inhibition for RAP-1
cELISA, 1 + fluorescence in Mo7 and/or T2Bo IFA.

One hundred seventy-seven of 228 sera collected from cattle herds in Australia that were vaccinated with an attenuated B. bovis strain or challenged with the pathogenic W strain were strong positive by IFA and the remaining 51 sera were weak positive. Analyses with all of these 228 sera resulted in 95.2% and 86.0% diagnostic sensitivity for the SBP4 MI-ELISA and the RAP-1 cELISA, respectively (Table 3). Thus, the SBP4 MI-ELISA had significantly (p≤0.004) better diagnostic sensitivity than the RAP-1 cELISA against these sera.

Three hundred eighty-eight of 598 IFA positive sera collected from endemic regions in Mexico were strongly IFA-positive and the rest were weak IFA-positive. The diagnostic sensitivity of the SBP4 MI-ELISA was 98.7% while diagnostic sensitivity of the RAP-1 cELISA was 60.0% for all 598 IFA-positive sera (Table 3). The SBP4 MI-ELISA has significantly (p<0.001) better diagnostic sensitivity than the RAP-1 cELISA for these sera.

The analytical specificity of the SBP4 MI-ELISA was evaluated for sera positive to B. bigemina (n=25) and Anaplasma marginale (n=25), which are other tick-borne intraerythrocytic parasites commonly co-infecting with B. bovis in cattle from endemic areas. All of these sera were negative in the SBP4 MI-ELISA, demonstrating suitable analytical specificity against closely related and/or frequently co-infecting intra-erythrocyte parasites (Tables 4 and 5).

TABLE 4
Analytical specificity of the SBP4 MI-ELISA evaluated with Babesia
bigemina antibody-positive sera.

| | B. bovis | | B. bigemina | |
|---|---|---|---|---|
| | SBP4 MI-ELISA* | | RAP-1 cELISA** | |
| Sample ID | S/N ratio | Result | % inhibition | Result |
| 1 | 1.1 | − | 66.4 | + |
| 2 | 1.9 | − | 51.3 | + |
| 3 | 1.8 | − | 57.6 | + |
| 4 | 1.3 | − | 46.6 | + |
| 5 | 1.6 | − | 54.9 | + |
| 6 | 1.6 | − | 54.9 | + |
| 7 | 1.1 | − | 56.9 | + |
| 8 | 1.9 | − | 51.1 | + |
| 9 | 1.6 | − | 50.5 | + |
| 10 | 1.3 | − | 44.9 | + |
| 11 | 1.3 | − | 67.6 | + |
| 12 | 1.2 | − | 77.6 | + |
| 13 | 1.1 | − | 80.2 | + |
| 14 | 1.2 | − | 81.8 | + |
| 15 | 1.1 | − | 81.9 | + |
| 16 | 1.2 | − | 80.4 | + |
| 17 | 1.2 | − | 78.4 | + |
| 18 | 1.1 | − | 80.0 | + |
| 19 | 1.1 | − | 76.6 | + |
| 20 | 1.3 | − | 65.3 | + |
| 21 | 1.1 | − | 79.8 | + |
| 22 | 1.1 | − | 61.7 | + |
| 23 | 1.1 | − | 56.4 | + |
| 24 | 1.2 | − | 52.5 | + |
| 25 | 1.1 | − | 54.0 | + |
| B. bovis (+) | 7.6 | + | NA | NA |
| B. bovis (−) | 1.0 | − | NA | NA |
| B. bigemina (+) | NA | NA | 55.3 | + |
| B. bigemina (−) | NA | NA | 0.0 | − |

*Positive cut-off ≥3 S/N ratio
**Positive cut-off ≥21% inhibition.

TABLE 5
Analytical specificity of the SBP4 MI-ELISA evaluated with
Anaplasma marginale antibody-positive sera.

| | B. bovis SBP4 | | A. marginale MSP5 | |
|---|---|---|---|---|
| | MI-ELISA* | | cELISA** | |
| Sample ID | S/N ratio | Result | % inhibition | Result |
| 1 | 0.7 | − | 93.4 | + |
| 2 | 1.1 | − | 91.7 | + |
| 3 | 1.1 | − | 93.6 | + |
| 4 | 1.6 | − | 48.2 | + |
| 5 | 2.7 | − | 74.9 | + |
| 6 | 2.2 | − | 80.3 | + |
| 7 | 2.2 | − | 83.1 | + |
| 8 | 2.1 | − | 82.7 | + |
| 9 | 2.3 | − | 85.6 | + |
| 10 | 2.2 | − | 85.2 | + |
| 11 | 1.8 | − | 89.1 | + |
| 12 | 1.7 | − | 87.2 | + |
| 13 | 2.0 | − | 88.2 | + |
| 14 | 1.6 | − | 88.1 | + |
| 15 | 1.5 | − | 86.9 | + |
| 16 | 1.5 | − | 87.1 | + |
| 17 | 1.2 | − | 94.5 | + |
| 18 | 1.1 | − | 89.0 | + |
| 19 | 1.6 | − | 91.0 | + |
| 20 | 1.3 | − | 93.0 | + |
| 21 | 1.8 | − | 90.8 | + |
| 22 | 1.1 | − | 87.4 | + |
| 23 | 1.3 | − | 91.9 | + |
| 24 | 2.1 | − | 84.5 | + |
| 25 | 1.8 | − | 81.9 | + |
| B. bovis (+) | 4.4 | + | NA | NA |
| B. bovis (−) | 1 | − | NA | NA |
| Anaplasma | NA | NA | 81.7 | + |
| (+) | | | | |
| Anaplasma (−) | NA | NA | 0 | − |

*Positive cut-off ≥3 S/N ratio
**Positive cut-off ≥30% inhibition

The results of the present study demonstrate that the SBP4 MI-ELISA has very high diagnostic specificity and sensitivity when evaluated with diverse sera collected from bovine herds in non-endemic, endemic, and epidemic areas. This SBP4 MI-ELISA can consistently detect B. bovis-specific antibodies in calves with acute as well as long-term carrier infections making it a pivotal tool for controlling B. bovis infection in cattle in endemic areas and preventing their movement to non-endemic and free areas.

Data analysis: The diagnostic specificities of the SBP4 MI-ELISA and the RAP-1 cELISA were calculated as the percentage of IFA-negative sera for B. bovis that were also negative by the assay in question. Diagnostic sensitivity was the percentage of the IFA-defined B. bovis-positive sera having a positive result in the assay being evaluated. Receiver operating characteristic (ROC) curve and scatter plot analysis were performed using spreadsheet software (Excel software, Microsoft, Seattle, Wash., USA) and R software from the R Foundation for Statistical Computing (Hornik 2016. “The R FAQ”, Retrieved from the internet: CRAN.R-project.org/doc/FAQ) to evaluate the cutoff for positive and negative detection by the newly developed SBP4 MI-ELISA through comparison with the IFA-positive and -negative serum reference panels described above (Greiner, M. 1995. J. Immunol. Methods 185:145-146; Greiner et al. 1995. J. Immunol. Methods 185:123-132; Griner et al. 1981. Ann. Intern. Med. 94:557-592).

Statistical analysis: Binomial test (Newcombe, R. G. 1998. Stat. Med. 17:873-890) was used to determine if there were significant (p<0.05) differences in sensitivity and specificity between the SBP4 MI-ELISA and RAP-1 cELISA or IFA test when testing various sets of serum samples. All statistical analyses were performed using R software described above.

### Example 7

### Infection of Cattle with Babesia bovis Strains; Parasitemia and Antibody Response

In a previous study (Suarez et al. 2012, supra) showing strong evidence of the long-term persistence of B. bovis in cattle, two calves were infected with the B. bovis Mo7 attenuated strain (Rodriguez et al. 1983. Infect. Immun. 42:15-18) and two calves were infected with the B. bovis Tf-137-4 strain (Hines et al. 1989. Mol. Biochem. Parasitol. 37:1-9; Levy and Ristic. 1980. Science 207:1218-1220). These four calves received a low dose (5×10³) of infected erythrocytes intravenously and were monitored for signs of acute babesiosis, including parasitemia, fever, and low packed cell volume (PCV) on a daily basis for the first twenty days post-inoculation (DPI) and on a biweekly basis thereafter until 11 months post-inoculation.

Two calves were infected with a high dose (5×10⁵) of erythrocytes infected with either the B. bovis Mo7 attenuated strain or the B. bovis T2Bo pathogenic strain (Suarez et al. 2006. Int. J. Parasitol. 36:965-973) and then were monitored as above on a daily basis for the first fifty DPI followed by a biweekly basis thereafter until 10 months post-inoculation. All six experimentally-infected animals had recurrent parasitemia lasting more than 10 months post-infection, even those infected with a low dose of attenuated B. bovis strains, indicating persistent infection capacity of all three B. bovis strains regardless of pathogenicity and challenge dose. Five animals challenged with attenuated strains had no clinical signs or thrombocytopenia after the first acute parasitemia. However, the calf challenged with the pathogenic B. bovis (T2Bo strain) had consistent parasitemia, thrombocytopenia and retarded weight gain during the 12 month monitoring period (data not shown). The consistent parasitemia in the calf challenged with pathogenic strain, T2Bo, and the significantly lower frequency of parasitemia in calves challenged with the attenuated strains, Mo7 and Tf-137-4, suggest lack of reliability of parasitemia detection-based diagnosis, e.g., PCR, due to the narrow window for detection of parasitemia in unapparent carriers.

Here, in this study, long-term duration and kinetics of parasitemia and B. bovis-specific antibody response are systematically defined in calves infected with high and low doses of three different B. bovis strains including attenuated and pathogenic ones. The protocol of infection used in these animal studies and all animal handling was approved by the Institutional Animal Care and Use Committee (IACUC) in Washington State University (#03735-008 approved on 12/9/2009).

DNA Extraction and PCR:

DNA was extracted with a purification reagent from FTA cards spotted with whole blood of test according to the manufacturer's instructions (FTA card and DNA purification reagent, GE Healthcare, Piscataway, N.J., USA). Briefly, blood samples were dotted on FTA cards and treated with a purification reagent to lyse erythrocytes and remove cellular proteins. DNA of each sample was eluted in PCR buffer and nested PCR to detect B. bovis DNA was performed according to previously reported methods (Suarez et al. 2012, supra).

Interestingly, all six calves maintained robust B. bovis antibody responses during all 12 months of the monitoring period when analyzed by the low throughput and relatively subjective sero-diagnostic tool, IFA. Persistence of antibody responses specific for B. bovis even at DPIs with no detectable parasitemia clearly indicates the relative advantage of an antibody-based diagnostic over an antigen detection assay, particularly when an accurate, high-throughput serology assay is available and results are interpreted more objectively than IFA results.

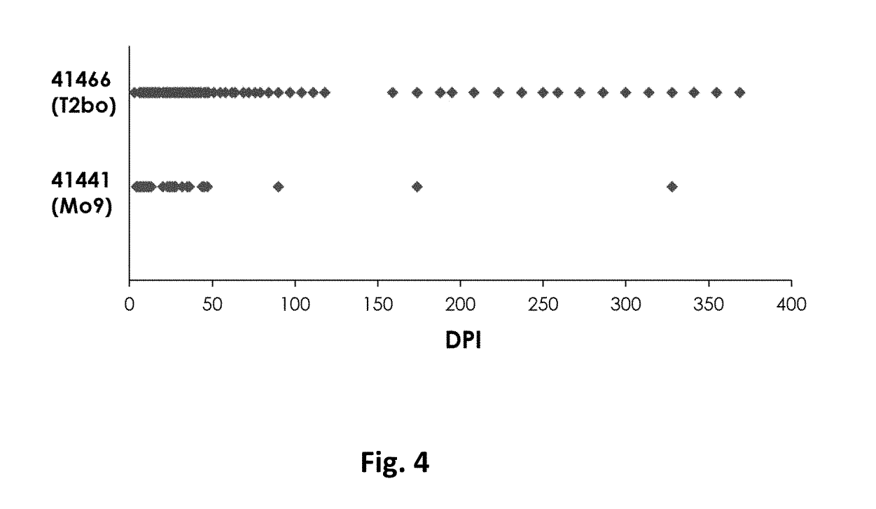

One calf was experimentally infected with a high dose (5×10⁵ infected erythrocytes) of B. bovis Mo7 and another calf was infected with 5×10⁵ erythrocytes infected with the T2Bo strain. Both had detectable parasitemia longer than 12 months when tested daily for the first 33 days post-infection and biweekly until 12 months post-inoculation by nested PCR (FIG. 4). Initial parasitemia detected by PCR in these two calves was four days post-infection (DPI) (FIG. 4). Calf C41466, infected with the T2Bo strain, had more frequent positive nested PCR results than calf C41441, infected with the cloned moderately-attenuated Mo7 strain. After the initial positive PCR at four DPI, there were 61 (81.3%) versus 25 (33.3%) PCR positive time points out of 75 tested for C41466 and C41441, respectively (FIG. 4).

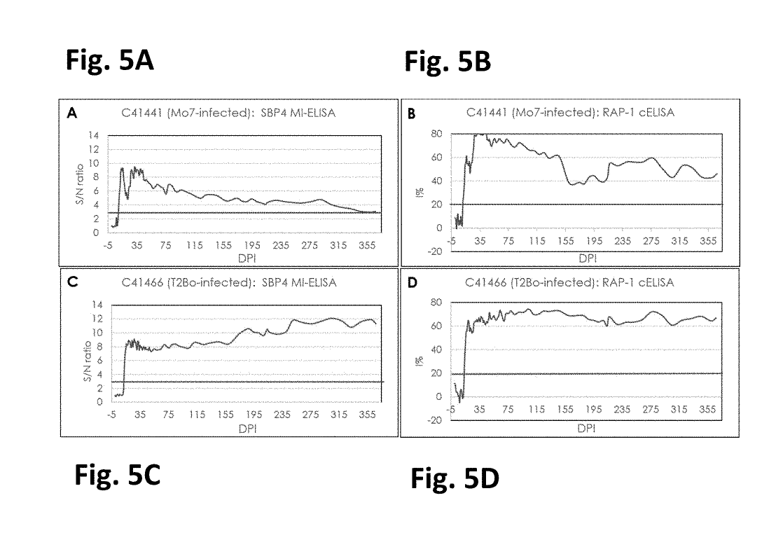

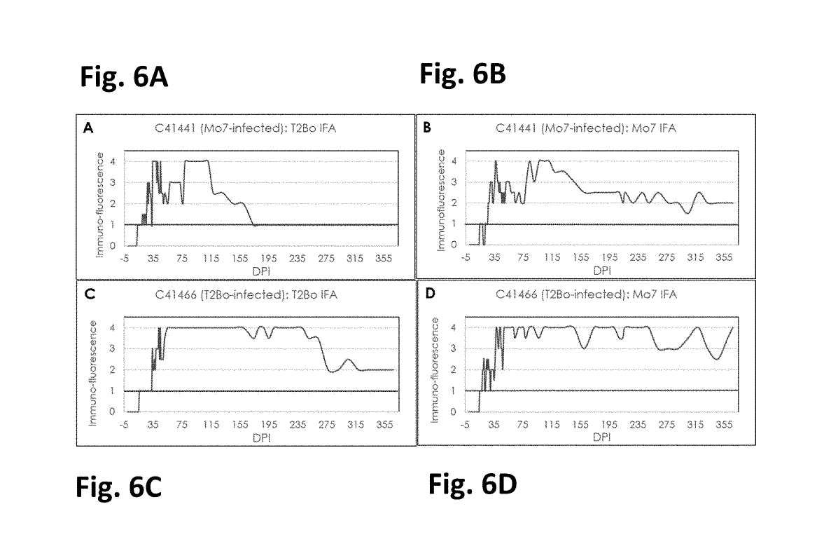

The timing of initial antibody detection in experimentally-infected calves was slightly variable depending on the assay and the B. bovis strain used for infection. The calf infected with a high dose of moderately attenuated Mo7 strain had detectable antibody beginning at 10, 11 and 14 days DPI by the SBP4 MI-ELISA (FIG. 5A), the RAP-1 cELISA (FIG. 5B) and IFA (FIGS. 6A and 6B), respectively. The calf infected with a high dose of T2Bo strain had detectable antibody beginning at 13, 14 and 15 DPI by the same three assays, respectively (FIGS. 5C, 5D, 6C and 6D). Thus, the difference between the SBP4 MI-ELISA and the RAP-1 cELISA in the timing of initial antibody detection was only one day for both strains.

B. bovis-specific antibody responses after initial positive detections were maintained for longer than 12 months post-infection according to all three assays (FIGS. 5 and 6). The long-term pattern of antibody responses in the T2Bo-infected calf C41466 was consistently robust and increased for 12 months with only minor fluctuations as assessed by the SBP4 MI-ELISA (FIG. 5C). Positive antibody responses were observed at all of 66, 65 and 65 time points tested after the first positive detection by SBP4 MI-ELISA, RAP-1 cELISA and IFA, respectively (FIGS. 5 and 6). Using time points collected in C41466 after the first positive result from SBP4 MI-ELISA, the diagnostic sensitivities of nested PCR, SBP4 MI-ELISA, RAP-1 cELISA and IFA were 87.9%, 100%, 98.5% and 97.0%, respectively. However, the antibody response detected by the SBP4 MI-ELISA in calf C41441 (challenged with the attenuated Mo7 strain) was less robust and gradually decreased to near the S/N ratio cutoff at 12 months post-infection (FIG. 5A). A similar pattern was observed using the RAP-1 cELISA (FIG. 5B) and IFA (FIG. 6B). C41441 had positive time points at all of 69, 68 and 66 time points tested after the first positive detection by SBP4 MI-ELISA, RAP-1 cELISA and IFA, respectively (FIGS. 5 and 6). Using time points collected in C41441 after the first positive result in SBP4 MI-ELISA, the diagnostic sensitivities of nested PCR, SBP4 MI-ELISA, RAP-1 cELISA and IFA were 27.5%, 100%, 98.6% and 94.2%, respectively. These results demonstrate that the three antibody assays, particularly SBP4 MI-ELISA, have higher diagnostic sensitivity than the nested PCR in monitoring infection status in these calves experimentally infected with a high dose of B. bovis.

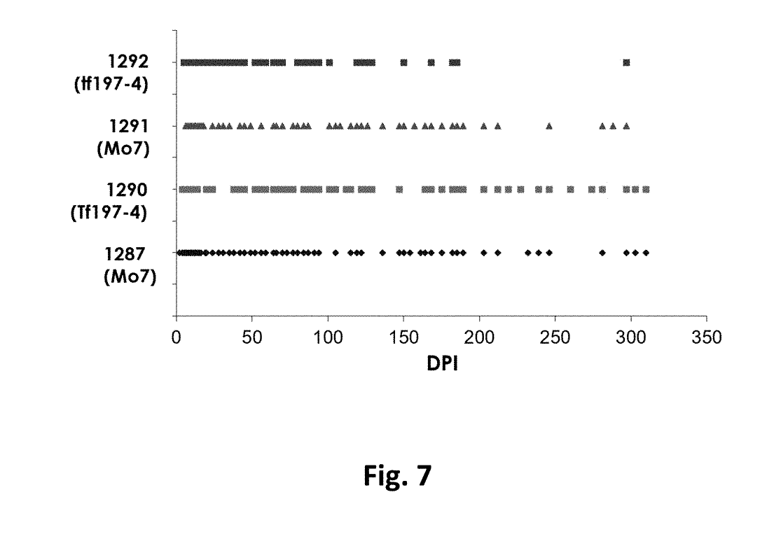

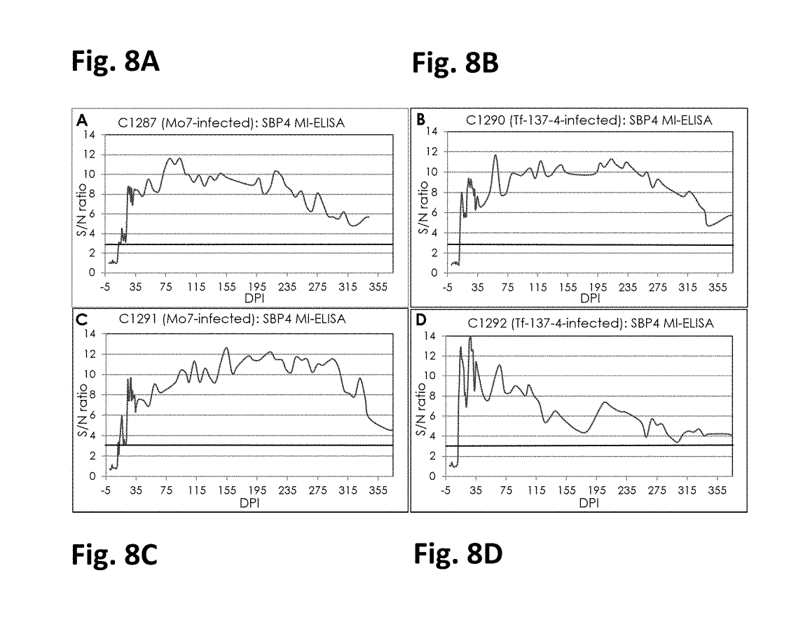

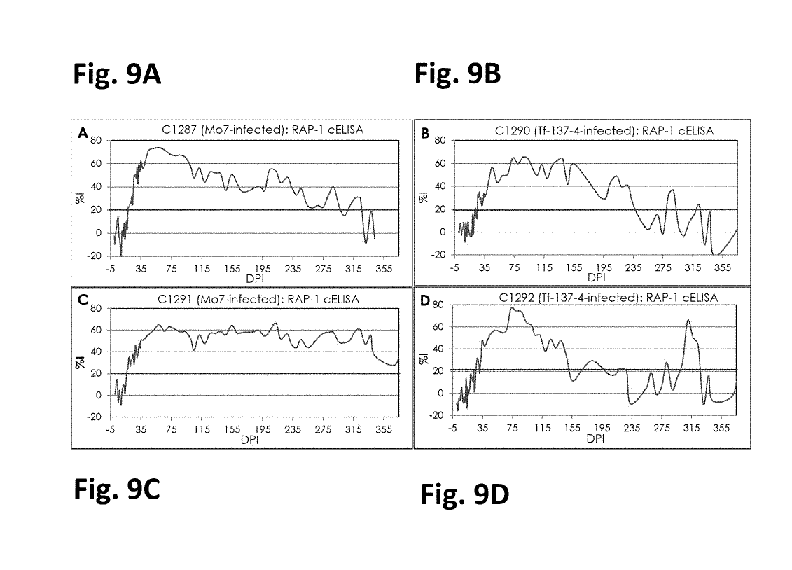

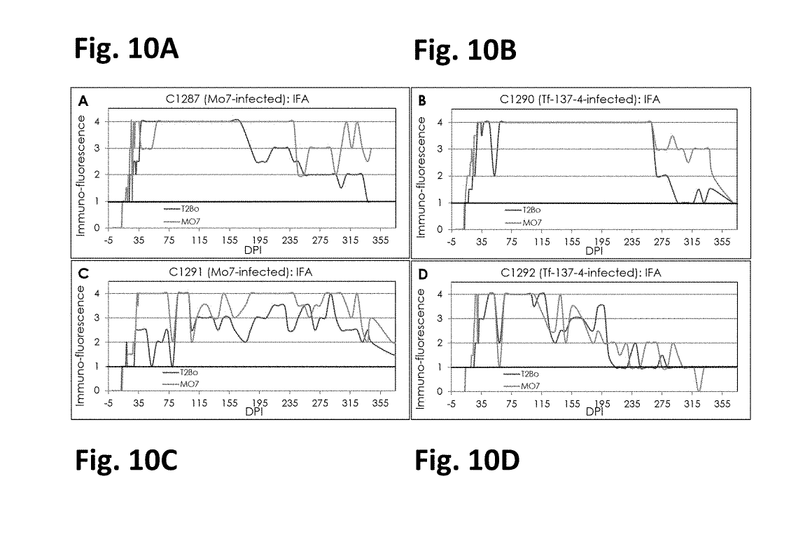

Two calves experimentally infected with a low dose (5×10³ infected erythrocytes) of B. bovis Mo7 strain (C1287 and C1291) and two calves infected with the same number of erythrocytes infected with the attenuated Tf-137-4 strain (C1290 and C1292) had variable parasitemia for longer than 11 months when tested by the nested PCR, demonstrating long-term persistence of B. bovis after infection (FIG. 7). Initial parasitemia detection by nested PCR in these four calves was 2 to 6 DPI (FIG. 7). The timing of initial antibody detection in low dose B. bovis-infected calves was notably variable, being both assay- and strain-dependent (FIGS. 8, 9 and 10). The two calves infected with an attenuated B. bovis strain, Tf-137-4, had detectable B. bovis-specific antibodies at 12 and 14-15 DPI when tested by SBP4 MI-ELISA (FIGS. 8B and 8D) and IFA (FIGS. 10B and 10D). However, the first positive antibody detection by the RAP-1 cELISA in the same calves was at 26 DPI, representing an 11 to 14 day delay in detection in calves infected with a low dose of an attenuated strain (FIGS. 9B and 9D). The two Mo7-infected calves had detectable antibody at 13 and 13-14 DPI when tested by the SBP4 MI-ELISA (FIGS. 8A and 8C) and IFA (FIGS. 10A and 10C), respectively. However, the earliest positive antibody detection in the same calves was at 18 DPI using the RAP-1 cELISA (FIGS. 9A and 9C), a four to five day delay in detection.

Following initial detection in these four calves, the long-term pattern of parasitemia was variable, as was B. bovis-specific antibody responses detected by all three serologic assays. After the first positive SBP4 MI-ELISA result at 13 DPI, the Mo7-infected calves had 84.7% (50 of 59 time points) and 69.5% (41 of 59) PCR-positive time points (FIG. 7). Using the same time points, 100% were SBP4 MI-ELISA positive (FIG. 8), 83.1% to 86.4% were RAP-1 cELISA positive (FIG. 9), and 98.3 to 100% were positive with IFA (FIG. 10). Tf-137-4-infected calves had 78.3% (47 of 60 time points) and 56.7% (34 of 60) PCR-positive time points after 12 DPI, the first positive SBP4 MI-ELISA result (FIG. 7). Using the same time points, 100%, 50.0 to 55.0% and 95.0 to 96.7% were antibody positive by SBP4 MI-ELISA (FIG. 8), RAP-1 cELISA (FIG. 9) and IFA (FIG. 10), respectively. Positive antibody responses after initial detection in all four calves were maintained at all time points up to 12 months when tested by the SBP4 MI-ELISA (FIG. 8). Following a one to three day delay in antibody detection as compared to SBP4 MI-ELISA, IFA also had persistent positive detection at all time points (FIG. 10). However, B. bovis-specific antibodies in three of the same four calves dropped rapidly below the cutoff by 155 DPI in CC1292, 240 DPI in C1290 and 303 DPI in C1287 when tested by the RAP-1 cELISA (FIGS. 9A, 9B and 9D), suggesting insufficient reliability of the RAP-1 cELISA for monitoring the inapparent carrier stage of B. bovis infection.

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

1-3: (canceled)

4: A cDNA molecule encoding a recombinant rGST-SBP4 fusion protein comprising glutathione S-transferase (GST) and spherical body protein-4 (SBP4) antigen of Babesia bovis,
wherein the SBP4 has been modified by having the signal sequence for SBP4 deleted.

5: A method of detecting antibodies to Babesia bovis in an individual, the method comprising the steps of:
(a) contacting a biological sample from said individual with a rGST-SBP4 fusion protein antigen for a time and under conditions sufficient to form antigen/antibody complexes; and
(b) detecting in the biological sample the presence of antibodies that bind to the rGST-SBP4 fusion protein antigen, thereby detecting B. bovis infection in said individual,
wherein the rGST-SBP4 fusion protein comprises glutathione S-transferase (GST) and spherical body protein-4 (SBP4) antigen of Babesia bovis, and
wherein the SBP4 has been modified by having the signal sequence for SBP4 deleted.

6: The method of claim 5, wherein said biological sample is selected from the group consisting of blood, serum, plasma, saliva, cerebrospinal fluid, milk, colostrum and urine.

7: The method of claim 5, wherein the recombinant rGST-SBP4 fusion protein antigen is bound, conjugated or immobilized on or to a solid support.

8: The method of claim 7 wherein said solid support is a glutathione-bovine serum albumin (BSA)-coated solid support, said glutathione-BSA ensuring improved presentation quality of epitopes wherein GST of said rGST-SBP4 fusion protein antigen binds to said glutathione of glutathione-BSA.

9: The method of claim 8 wherein said rGST-SBP4 bound to said glutathione-BSA-coated support can be prepared and then stored at 4° C.

10: The method of claim 8 wherein said solid support is an immunoassay plate.

11: The method of claim 5 or claim 8 wherein the detecting step further comprises adding, after said contacting step, an indicator reagent comprising a reporter group conjugated to the rGST-SBP4 fusion protein.

12: The method of claim 11, wherein the reporter group is selected from the group consisting of radioisotopes, fluorescent groups, luminescent groups, enzymes, biotin and dye particles.

13: The method of claim 11 further comprising comparing the level of antibody specific for SBP4 antigen of B. bovis to control levels, wherein a level of anti-SBP4 antigen of B. bovis above the control is indicative of B. bovis infection.

14: A method for detecting infection of Babesia bovis strains in an individual, the method comprising a modified indirect ELISA comprising:
(a) treating an 96-well immunoassay plate with glutathione-bovine serum albumin and incubating said plates overnight at 4° C.,
(b) adding 200 μl/well of blocking buffer, then incubating for 2 hours at 37° C.,
(c) removing the blocking buffer and allowing plates to dry overnight,
(d) adding 50 μl/well of a dilution of the rGST-SBP4 fusion protein antigen that gave approximately 0.08 optical density at 450 nm (OD450) when tested against the negative reference serum to the prepared 96-well plates,
(e) storing the antigen-coated plates individually in polyester film bags at 4° C. until used,
(f) adding 50 μl/well of a test biological sample to wells of said immunoassay plates and incubating said plates at room temperature for 30 minutes,
(g) washing wells three times with 250 μl of wash buffer per well,
(h) adding 50 μl of HRP-conjugated rGST-SBP4 diluted in conjugate diluting buffer to each well of said immunoassay plates and incubating said plates at room temperature for 30 min,
(i) washing said immunoassay plates three times with 250 μl of wash buffer per well,
(j) adding 50 μl of tetramethylbenzidine substrate to each well and incubating said plates at room temperature for 15 min,
(k) stopping the reactions with 50 μl of 1.5% sodium fluoride solution per well, and
(l) obtaining an S/N ratio of sample optical density (OD) to negative control OD from said OD of the assay wells read at OD450, wherein a 3 S/N ratio is indicative of a positive result and detection of infection with Babesia bovis in said individual.

15: A kit for detecting infection with Babesia bovis in a biological sample from an individual, comprising:
(a) a rGST-SBP4 fusion protein,
(b) HRP-conjugated rGST-SBP4 detection conjugate,
(c) a set of positive and negative control sera, and
(d) an instruction for coating the support with glutathione-BSA to improve the presentation quality of SBP4 epitopes, followed by addition of the rGST-SBP4 fusion protein antigen resulting in oriented steric presentation of poly SBP4 B. bovis epitopes, followed by addition of the biological sample whereby the binding of antibodies specific for said SBP4 epitopes in said biological sample results in the formation of an antigen-antibody immunological complex, followed by addition of the HRP-conjugated rGST-SBP4 detection conjugate to detect presence or absence of an immunological complex, whereby presence of an immunological complex is indicative of infection with B. bovis in said biological sample,
wherein the rGST-SBP4 fusion protein comprises glutathione S-transferase (GST) and spherical body protein-4 (SBP4) antigen of Babesia bovis, and
wherein the SBP4 has been modified by having the signal sequence for SBP4 deleted.
